(19) United States
(12) Patent Application Publication
(10) Pub. No.: US 2024/0140988 A1
(43) Pub. Date: May 2, 2024

(54) SCALED-UP METHODS FOR PURIFYING ANTIBODIES

(71) Applicants: Ariel Scientific Innovations Ltd., Ariel (IL); Yeda Research and Development Co. Ltd., Rehovot (IL)

(72) Inventors: Guy PATCHORNIK, Kiryat-Ono (IL); Mordechai SHEVES, Rehovot (IL); Irishi N. N. NAMBOOTHIRI, Mumbai (IN); Gunasekaran DHANDAPANI, Ariel (IL)

(73) Assignees: Ariel Scientific Innovations Ltd., Ariel (IL); Yeda Research and Development Co. Ltd., Rehovot (IL)

(21) Appl. No.: 17/637,098

(22) PCT Filed: Apr. 16, 2020
(86) PCT No.: PCT/IL2020/050452

§ 371 Date: Feb. 22, 2022

Related U.S. Application Data
(60) Provisional application No. 62/924,204, filed on Oct. 22, 2019.

(30) Foreign Application Priority Data
Aug. 22, 2019 (IL) 268878

(51) Int. Cl. C07K 1/32; C07K 1/34

(52) U.S. Cl. CPC C07K 1/32; C07K 1/34; C07K 2317/14

(57) ABSTRACT

A method of isolating an antibody is disclosed. The method comprises contacting a hydrophobic chelator, a non-ionic detergent and metal ions so as to generate an aggregate comprising the hydrophobic chelator, the detergent and the metal ions; and contacting the aggregate with a medium comprising the antibody under conditions that allow partitioning of the antibody into the aggregate. Kits for isolating the antibody are also disclosed.

### RELATED APPLICATIONS

This application claims the benefit of priority of Israel Application No. 268878 filed 22 Aug. 2019 and U.S. Provisional Patent Application No. 62/924,204 filed 22 Oct. 2019, the contents of which are incorporated herein by reference in their entirety.

### FIELD AND BACKGROUND OF THE INVENTION

The present invention, in some embodiments thereof, relates to methods and, kits for purifying antibodies.

Monoclonal antibodies (mAb's) are currently the recombinant proteins most commonly used as therapeutics; they were the largest selling class of biologics in the USA in 2012. The dramatic increase in their expression levels from low milligram to multi-gram concentration per liter, together with the multi-hundred kilogram to ton quantities in which some of them will be required, pose an on-going challenge for industrial purification methods capable of efficiently capturing mAb's from complex mixtures. This is generally achieved via ProA chromatography as the initial capturing step, commonly resulting in high recovery yields (˜95%), purity (>95%), while removing the majority of host DNA, viral contaminants and leached ProA.

These remarkable features have made ProA chromatography the gold standard for antibody manufacturing. However, there is motivation for the development of more economic alternatives since ProA resins suffer from high costs relative to non-affinity polymeric supports (e.g. ion exchangers). This motivation is further justified when considering the current and future global biotech demands (i.e. many tons of purified mAb's per year) representing hundreds of different therapeutic mAb's under development, all aimed at targeting various cancers, autoimmune and inflammatory disorders.

It has been argued that, the use of ProA, and of chromatographic strategies in general, represent an inherent “productivity bottleneck” for industrial purification of mAb's, which can account for up to 80% of the total manufacturing cost thus making any antibody capturing method not entailing: (a) ProA as a ligand and/or (b) chromatography as the primary capturing step, an attractive alternative for future pharmaceutical needs.

Background art includes Patchornick et al., Bioconjugate Chemistry, 2013, Volume 24, pages 1270-1275; Guse et al., J. Chromatogr A. (1994) 661, 13-23; Manske et al., J. Immunol Methods (1997) 2008, 65-73; Follman and Fahrner J. Chromatogr A. (2004) 1024, 79-85 and Ghosh and Wang, J. Chromatogr A. (2006) 1107, 104-109.

Additional background art includes WO2018/207184.

### SUMMARY OF THE INVENTION

According to an aspect of the present invention there is provided a method of isolating an antibody, the method comprising:
(a) contacting a hydrophobic chelator, a non-ionic detergent and an iron salt, so as to generate an aggregate comprising the hydrophobic chelator, the detergent and iron ions of the iron salt, the iron salt being selected from the group consisting of iron chloride, iron bromide and iron fluoride; and
(b) contacting the aggregate with a medium comprising the antibody under conditions that allow partitioning of the antibody into the aggregate, thereby isolating the antibody.

According to an aspect of the present invention there is provided a method of isolating an antibody, the method comprising:
(a) contacting a hydrophobic chelator, a non-ionic detergent and metal ions so as to generate an aggregate comprising the hydrophobic chelator, the detergent and the metal ions;
(b) contacting the aggregate with a medium comprising the antibody under conditions that allow partitioning of the antibody into the aggregate; and subsequently
(c) filtering the medium comprising the aggregate, thereby isolating the antibody.

According to an aspect of the present invention there is provided a method of preparing an aggregate, the method comprising:
(a) contacting a hydrophobic chelator, a non-ionic detergent and metal ions so as to generate a first aggregate comprising the hydrophobic chelator, the detergent and the metal ions;
(b) contacting the aggregate with a medium comprising an antibody under conditions that allow a first fraction of the antibody to partition into the aggregate;
(c) isolating the antibody from the aggregate;
(d) disassociating the aggregate;
(e) isolating the hydrophobic chelator; and subsequently
(f) contacting the hydrophobic chelator, a non-ionic detergent and metal ions so as to generate a second aggregate comprising the hydrophobic chelator, the detergent and the metal ions, thereby preparing the aggregate.

According to an aspect of the present invention there is provided a kit comprising a hydrophobic chelator, a non-ionic detergent, a buffer having a pH between 3-6 and an iron salt selected from the group consisting of iron chloride, iron bromide and iron fluoride.

According to an aspect of the present invention there is provided a kit comprising a hydrophobic chelator, a polysorbate surfactant and an iron salt selected from the group consisting of iron chloride, iron bromide and iron fluoride.

According to embodiments of the invention, the disassociating the aggregate comprises contacting the aggregate with a water soluble chelator under conditions that allows disassociation of the aggregate.

According to embodiments of the invention, the method further comprises filtering the medium comprising the aggregate.

According to embodiments of the invention, the iron salt is iron chloride.

According to embodiments of the invention, the water soluble chelator comprises EDTA or EGTA.

According to embodiments of the invention, the isolating the antibody from the aggregate comprises filtering the medium comprising the aggregate.

According to embodiments of the invention, the aggregate has a diameter of greater than 500 nM.

According to embodiments of the invention, the aggregate has a diameter of between 500-3000 nM.

According to embodiments of the invention, the medium comprises a cell lysate.

According to embodiments of the invention, the cell lysate is a whole cell lysate.

According to embodiments of the invention, the medium comprises a hybridoma medium.

According to embodiments of the invention, the medium comprises serum albumin.

According to embodiments of the invention, the cell lysate is devoid of organelles greater than about 2 microns.

According to embodiments of the invention, the conditions of step (b) comprise having a level of salt below 100 mM.

According to embodiments of the invention, the method further comprises solubilizing the antibody following step (b).

According to embodiments of the invention, the isolating the antibody comprises solubilizing the antibody.

According to embodiments of the invention, the solubilizing is effected with a buffer having a pH between 3-6.

According to embodiments of the invention, the solubilizing is effected with a buffer having a pH between 3.8 and 4.

According to embodiments of the invention, the buffer further comprises a salt.

According to embodiments of the invention, the buffer is a carboxylic buffer.

According to embodiments of the invention, the buffer comprises an amino acid.

According to embodiments of the invention, the carboxylic buffer is selected from the group consisting of isoleucine, valine, glycine and sodium acetate.

According to embodiments of the invention, the non-ionic detergent is a polysorbate surfactant.

According to embodiments of the invention, the polysorbate surfactant is selected from the group consisting of polysorbate 20, polysorbate 40, polysorbate 60 and polysorbate 80.

According to embodiments of the invention, the hydrophobic chelator comprises 8-Hydroxyquinoline.

According to embodiments of the invention, the hydrophobic chelator comprises a phenanthroline.

According to embodiments of the invention, the phenanthroline is selected from the group consisting of N-(1,10-Phenanthrolin-5-yl)methanamide) (Phen-C1), N-(1,10-Phenanthrolin-5-yl)ethan amide) (Phen-C2), N-(1,10-Phenanthrolin-5-yl)propanamide) (Phen-C3), N-(1,10-Phenanthrolin-5-yl)butanamide) (Phen-C4), N-(1,10-Phenanthrolin-5-yl)pentanamide) (Phen-05), N-(1,10-Phenanthrolin-5-yl)hexanamide) (Phen-C6), N-(1,10-Phenanthrolin-5-yl)heptanamide) (Phen-C7), N-(1,10-Phenanthrolin-5-yl)octanamide) (Phen-C8), N-(1,10-Phenanthrolin-5-yl)nonanamide) (Phen-C9) and N-(1,10-Phenanthrolin-5-yl)decanamide) (Phen-C10).

According to embodiments of the invention, the phenanthroline is selected from the group consisting of bathophenanthroline, N-(1,10-Phenanthrolin-5-yl)hexanamide) (Phen-6), N-(1,10-Phenanthrolin-5-yl)decanamide) (Phen-C10) and N-(1,10-Phenanthrolin-5-yl)octanamide) (Phen-C8).

According to embodiments of the invention, the phenanthroline is bathophenanthroline.

According to embodiments of the invention, the metal ions are divalent metal ions.

According to embodiments of the invention, the divalent metal ions are selected from the group consisting of Zn²⁺, Fe²⁺, Mn²⁺, Ni²⁺ and Co²⁺.

According to embodiments of the invention, the divalent metal ions are selected from the group consisting of Zn²⁺ and Fe²⁺.

According to embodiments of the invention, the hydrophobic chelator is present in the medium at a concentration in the range of about 0.1% to about 10% (v/v).

According to embodiments of the invention, the metal ions are present in the medium at a concentration in the range of about 0.1% about 10% (v/v).

According to embodiments of the invention, the cell lysate is derived from a bacterial cell.

According to embodiments of the invention, the cell lysate is derived from a mammalian cell.

According to embodiments of the invention, the mammalian cell is a Chinese Hamster Ovary cell (CHO).

According to embodiments of the invention, the antibody is a humanized antibody.

According to embodiments of the invention, the antibody is a recombinant antibody.

According to embodiments of the invention, the antibody is selected from the group consisting of IgA, IgD, IgE, IgM and IgG.

According to embodiments of the invention, the IgG is IgG1, IgG2, IgG3 or IgG4.

According to embodiments of the invention, the Fe²⁺ is generated from a salt selected from the group consisting of iron chloride, iron bromide and iron fluoride.

According to embodiments of the invention, the Fe²⁺ is comprised in iron chloride.

According to another aspect of the present invention there is provided a method of isolating an antibody fragment of interest comprising:
(a) contacting a hydrophobic chelator, a non-ionic detergent and metal ions so as to generate an aggregate comprising the hydrophobic chelator, the detergent and the metal ions;
(b) contacting said aggregate with a medium comprising:
(i) an antibody fragment which comprises an Fc region; and
(ii) an antibody fragment which is devoid of an Fc region,
wherein said contacting is effected under conditions that allow selective partitioning of one of said fragments (i) or (ii) into said aggregate so as to generate an antibody fragment-enriched aggregate and an antibody fragment-enriched medium; and
(c) isolating the antibody fragment of interest from the fragment-enriched aggregate or from the fragment-enriched medium, thereby isolating the antibody fragment of interest.

According to embodiments of the invention, the conditions comprise a pH of between 5-9.

According to embodiments of the invention, the pH is between 7-8.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B: Yield of the engineered-micelle platform for Ab purification as compared to ProA or Protein G (ProG) resins. Centrifugation: Lanes 1-2: hIgG (control); lanes 3-4 and 5-6: supernatant composition obtained after purification of hIgG with Protein A or Protein G spin columns; Lanes 7-8: supernatant composition after purification of hIgG with Tween-20 detergent aggregates. Filtration: As in Centrifugation, but by applying filtration, i.e.: the BSA+IgG mixture is added to preformed Tween-20:bathophenanthroline:Fe2+] aggregates, incubated for 5 minutes and filtration is applied (0.22 micron filters). The resulting precipitate is washed with 30 mM NaCl and an extraction buffer is added. The system is further incubated for 2-30 minutes at 20-42° C. and filtration is applied again (0.22 micron filters). Lanes 5-6 show the composition of the filtrate obtained via this protocol. BSA, H & L are bovine serum albumin and the reduced heavy and light chains of the target antibody, respectively. Gels are Coomassie blue stained.

FIGS. 2A-D—Supernatant composition (after IgG capture & extraction) with indicated detergents, the: [Bathophenanthroline):Fe2+] complex and human IgG (hIgG) as the target. BSA, H & L are bovine serum albumin and the reduced heavy and light chains of the target antibody, respectively. Gels are Coomassie blue stained.

FIG. 3: Purification of human IgG (hIgG) in the presence of indicated chelators. Lanes 1-2: Control: Supernatant composition containing the recovered hIgG after its capture with: [Tween-20:bathophenanathroline:Fe2+:PEG-6000] aggregates and further extraction with 50 mM Ile at pH 3.8 (15 minutes at 32° C.); lanes 3-4, 5-6, 7-8, and 9-10 as in lanes 1-2, but with the presence of indicated chelators concentrations during the IgG capturing step. H, L denote the reduced heavy and light chains of the target antibody, respectively. Gels are Coomassie stained.

FIG. 4. Steps associated with chelator recycling. Step I: Removal of the water-soluble detergent (Tween-20) and antibodies. Step II: Dissociation of the red [(batho)₃:Fe²⁺] hydrophobic complex with excess of the water-soluble chelator: EDTA. Step III: Crystals observed at low temperature.

FIGS. 5A-B. A. Red crystals observed after washing Tween-20 aggregates with NaCl. B. Comparison in absorbance between freshly prepared: red [(batho)₃:Fe²⁺] complex and dissolved crystals shown in A.

FIGS. 6A-B. A. Mass spectrometry analysis of pure (control) batho crystals and B. regenerated colorless crystals shown in FIG. 4.

FIGS. 7A-B. Effect of metal anion (SO₄²⁻ vs. Cl⁻) on process yield and purity. SDS-PAGE analysis: Lane 1: Control—BSA (0.25 mg\ml) and human IgG (1 mg\ml) mixture; lanes 2-4: Recovered hIgG after IgG capture and extraction from aggregates generated in the presence of FeSO₄ as described in the Examples herein below, lanes 5-7: as in lanes 2-4 but the presence of FeCl₂. BSA, H, L denote bovine serum albumin, the reduced heavy and light chains of the target antibody, respectively. Gels are Coomassie stained. B. Temperature effect on IgG capture using Tween-20 aggregates. SDS-PAGE analysis: Lane 1: Control—human IgG (5 mg\ml) representing the total amount of antibody in the system; lanes 2-4: Amount of hIgG present in the supernatant after incubation at 22° C. using greater amount of Tween-20 aggregates (×2.5) in comparison to that used for the purification of 1 mg\ml hIgG as described in the Examples, herein below; lanes 5-7: As in lanes 2-4 but with lower amount of Tween-20 aggregates (×1.5); lanes 8-10: As in lanes 5-7 but at 4° C.; H, L denote the reduced heavy and light chains of the target antibody, respectively. Gels are Coomassie stained.

FIG. 8 is an illustration of the purification strategies of antibody fragments according to embodiments of the present invention.

FIGS. 9A-B. Digestion of a mAb with papain. Lane 1: reduced mAb; lane 2: non-reduced mAb; lane 3: Papain digested mAb. B. Supernatant composition after a brief incubation of indicated detergent aggregates with papain cleaved mAb. Both gels are Coomassie stained.

FIGS. 10A-D. Parameters affecting the purification of antibody fragments according to particular embodiments. A. Effect of indicated temperatures on the binding of Ab-fragments to Brij S-100 aggregates. Samples represent the supernatant composition after a brief incubation (10 min.) with the aforementioned aggregates. B. As in A, but at indicated pH values. C. As in A, but at indicated salt concentrations. D. As in A, but at pH 7.4, 13 mM NaCl and indicated time points. All gels are Coomassie stained.

FIGS. 11A-E. Parameters affecting the purification of antibody fragments according to particular embodiments. A. Effect of indicated temperatures on the extraction efficiency of Ab-fragments from Tween-60 aggregates at pH 3.8 and the presence of 50 mM Leucine and 125 mM NaCl. B. As in A, but at indicated pH values. C. As in A, but at indicated salt concentrations. D. As in A, but at pH 7.4, 13 mM NaCl and indicated time points. E. As in A, but with indicated buffers, salt after 10 minutes at room temperature. All gels are Coomassie stained.

FIG. 12 is a table summarizing overall yields of strategy II with a mAb and polyclonal human IgG (hIgG).

FIG. 13. Comparison of amino acid composition between the Fc & Fab domains in IgG's. Number of amino acids present in either the Fc or Fab domains capable of binding metals (LHS), being hydrophobic (center) or hydrophilic (RHS), is indicated. The results derive from published sequences of 10 different IgG's exhibiting diverse specificities.

### DESCRIPTION OF SPECIFIC EMBODIMENTS OF THE INVENTION

The present invention, in some embodiments thereof, relates to methods and, kits for purifying antibodies. In particular, the methods relate to an alternative route for antibody capturing without the use of the common ligand, Protein A (ProA).

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Purification of antibodies typically uses Protein A (proA) chromatography as the initial capturing step. However, proA chromatography is very expensive creating a “productivity bottleneck”.

The present inventors uncovered a new method of purifying antibodies based on the use of hydrophobic chelators, non-ionic detergents and metal ions (see WO2018/207184).

In order to adapt those methods to ones that could be used for large-scale antibody purification, the present inventors have now surprisingly shown that a filtration step can be used to replace the small-scale laboratory centrifugation step. Furthermore, the present inventors showed that the hydrophobic chelator could be recycled and used in a second step of purification thereby enhancing the yield of purified antibody per unit weight of hydrophobic chelator.

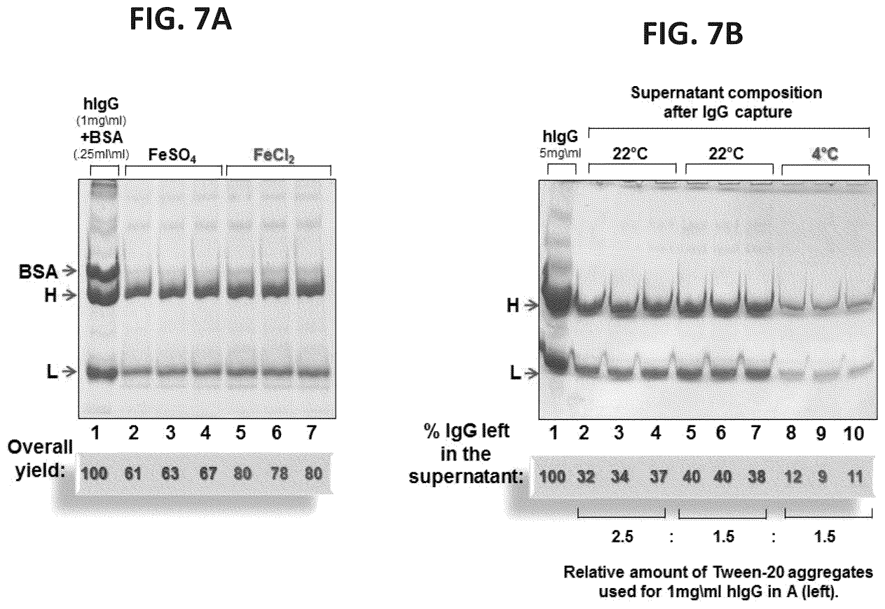

In addition, the present inventors have shown that surprisingly the anion of the metal salt influences the recovery yield of the antibody. Specifically, the present inventors showed that use of FeCl₂ as opposed to FeSO₄, significantly improved the yield of antibody and improvided the efficienty of the antibody capturing step at low temperatures (see FIGS. 7A-B).

Of note, unlike the use of Protein A as a general, highly efficient and robust ligand for antibody purification, no analogous general ligand exists for Ab-fragments, primarily due to the absence of the Fc domain onto which Protein A binds with high affinity and specificity. Thus, the absence of the Fc domain in Ab-fragments is tightly correlated with current difficulties associated with purification of this diverse class of proteins.

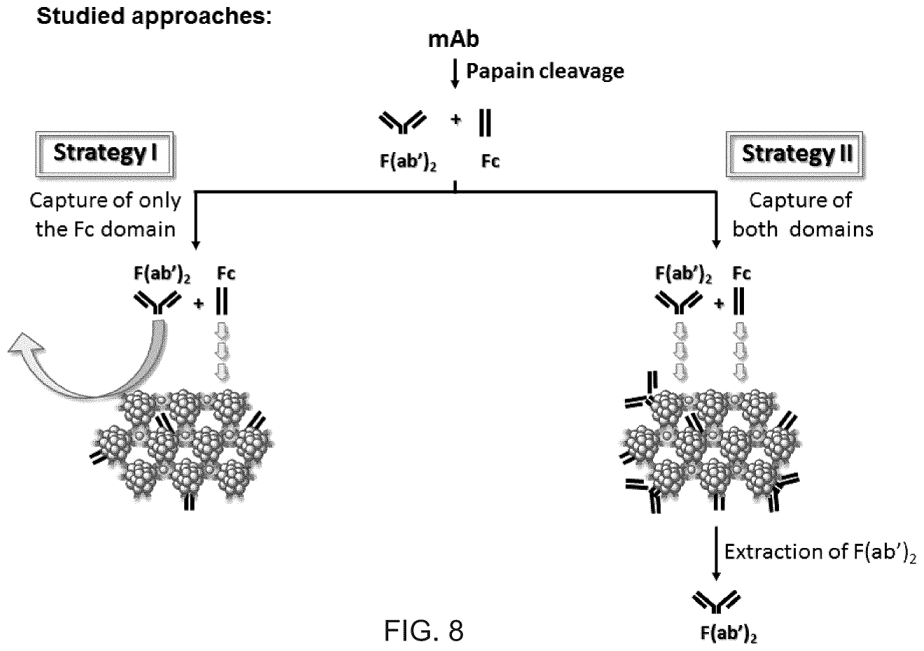

The present inventors have now found that the Fc domain of a papain digested mAb, binds stronger to detergent aggregates than the Fab or the F(ab′)₂ of the cleaved antibody. This discovery, paves the way towards the study of two independent purification strategies capable of separating the Fab and the F(ab′)₂ fragments from the Fc domain (as illustrated in FIG. 8).

Thus, according to a first aspect of the present invention there is provided a method of isolating an antibody, the method comprising:
(a) contacting a hydrophobic chelator, a non-ionic detergent and an iron salt, so as to generate an aggregate comprising the hydrophobic chelator, the detergent and iron ions of the iron salt, the iron salt being selected from the group consisting of iron chloride, iron bromide and iron fluoride; and
(b) contacting the aggregate with a medium comprising the antibody under conditions that allow partitioning of the antibody into the aggregate, thereby isolating the antibody.

The term “antibody” as used in this invention includes intact molecules as well as functional fragments thereof that comprise an Fc region and that are capable of binding to an epitope of an antigen.

The antibody of this aspect of the present invention may be a monospecific antibody or a bispecific antibody (i.e. capable of simultaneously binding two different or unique antigens).

Typically, the antibodies of this aspect of the present invention comprise at least one CDR in each of the variable regions.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et at, 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys™, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et at, Journal of Biological Chemistry, 283:1156-1166, 2008).

As used herein, the “variable regions” and “CDRs” may refer to variable regions and CDRs defined by any approach known in the art, including combinations of approaches.

In one embodiment, the antibody is a polyclonal antibody.

In another embodiment, the antibody is a monoclonal antibody.

In still a further embodiment, the antibody is a recombinant antibody.

In still a further embodiment, the antibody is a humanized antibody.

In still another embodiment, the antibody is an IgG.

In still further embodiments, the antibody is IgA, IgD, IgE and IgG (e.g. IgG1, IgG2, IgG3 or IgG4).

In still further embodiments, the antibody is IgM.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Humanized forms of non-human (e.g., murine) antibodies are chimeric molecules of immunoglobulins, immunoglobulin chains which contain minimal sequence derived from non-human immunoglobulin.

Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues.

Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)].

Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

When using recombinant techniques, the antibody can be produced intracellularly, in the periplasmic space, or directly secreted into the medium. If the antibody is produced intracellularly, as a first step, the particulate debris, either host cells or lysed cells, can be removed, e.g., by centrifugation or ultrafiltration. Where the antibody is secreted into the medium, supernatants from such expression systems can be first concentrated using a commercially available protein concentration filter, e.g., an Amicon™ or Millipore Pellicon™ ultrafiltration unit.

Lysis of the cells may be performed by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. Where the antibody is secreted, supernatants from such expression systems are generally first concentrated using a commercially available protein concentration filter, e.g., an Amicon™ or Millipore Pellicon™ ultrafiltration unit. Where the antibody is secreted into the medium, the recombinant host cells can also be separated from the cell culture medium, e.g., by tangential flow filtration.

In one embodiment, the medium in which the antibody is comprised is a cell lysate.

As used herein, the term “cell lysate” refers to an aqueous solution of cellular biological material which comprises the antibody, wherein a substantial portion of the cells of the cellular material have become disrupted and released their internal components.

In one embodiment, the cell lysate is prepared from whole cells.

In the case of a whole cell lysate, it will be appreciated that following cell membrane disruption, the cell lysate may be treated so as to remove organelles greater than about 2 microns (e.g. cell nucleii). Thus, for example the whole cell lysate may be centrifuged so as to precipitate cell nucleii from the cell lysate. Exemplary centrifugation conditions include 1-5 minutes at 500-1000×g (e.g. 2 min. at 985×g).

The cell lysate may be prepared from any cell that expresses an antibody. The cells may be eukaryotic (e.g. mammalian, plant, fungus) or prokaryotic (bacteria).

In another embodiment, the cells secrete antibody into the cell medium.

The cell may be genetically modified so as to express the antibody. In another embodiment, the cell is not genetically modified.

Exemplary cells that are contemplated include, but are not limited to gram negative bacterial cells, such as E. Coli; gram positive bacterial cells such as Bacillus brevis, Bacillus subtilis, Bacillus megaterium and Lactobacilli (e.g. Lactobacillus zeae/casei or Lactobacillus paracasei); yeast cells such as Pichia pastoris, Saccharomyces cerevisiae, Hansenula polymorpha, Schizosaccharomyces pombe, Schwanniomyces occidentalis, Kluyveromyces lactis, and Yarrowia lipolytica; filamentous fungii such as Trichoderma and Aspergillus; insect cells; mammalian cells including Chinese hamster ovary (CHO) cells and plant cells.

In one embodiment, the cells have been immortalized and are part of a cell line—e.g. hybridoma. As mentioned, the isolation method of this aspect of the present invention is carried out by contacting the medium comprising the antibody with aggregates of non-ionic detergent, hydrophobic chelator and metal ions.

Examples of cell media for culturing antibody producing cells include hybridoma media—e.g. serum-free hybridoma media. Such media are readily available from Companies such as Gibco, Thermo Fisher Scientific and Sigma-Aldrich.

In one embodiment, the media comprises a serum albumin such as horse serum albumin (HAS) or bovine serum albumin (BSA).

Preferably the serum albumin is present at a concentration of less than 0.5 mg/ml—for example between 0.1-0.5 mg/ml.

Prior to the isolation step, the medium comprising the antibody may optionally be clarified.

As used herein, the term “clarified” refers to a sample (i.e. a cell suspension) having undergone a solid-liquid separation step involving one or more of centrifugation, microfiltration and depth filtration to remove host cells and/or cellular debris. A clarified fermentation broth may be a cell culture supernatant. Clarification is sometimes referred to as a primary or initial recovery step and typically occurs prior to any chromatography or a similar step.

As mentioned the first step of the isolation comprises generation of an aggregate comprising a hydrophobic chelator, a non-ionic detergent and metal ions.

The term “non-ionic detergent” refers to detergents that comprise uncharged, hydrophilic headgroups. Some non-ionic detergents are based on polyoxyethylene or a glycoside. Common examples of the former include Tween, Triton, and the Brij series. These materials are also known as ethoxylates or PEGlyates and their metabolites, nonylphenol. Glycosides have a sugar as their uncharged hydrophilic headgroup. Examples include octyl thioglucoside and maltosides. HEGA and MEGA series detergents are similar, possessing a sugar alcohol as headgroup.

According to a particular embodiment, the non-ionic detergent is a polysorbate sufactant. Examples of such include, but are not limited to of polysorbate 20, polysorbate 40, polysorbate 60 and polysorbate 80.

In one embodiment, the non-ionic detergent is polysorbate 20.

Other exemplary non-ionic detergents contemplated by the present invention include those that belong to the pluronic family e. g. F-68 and F-127.

As used herein, the term “chelator” refers to a compound which binds metal ions from solution, by the formation or presence of two or more separate co-ordinate bonds between a polydentate ligand and a single central atom. The chelator of this aspect of the present invention is capable of chelating the metal ion which is used for the isolation. Preferably, the chelator binds electrostatically (non-covalently) to the metal ion. According to a particular embodiment, the chelator is capable of chelating metal ions with a ratio of chelator to metal of 2:1 or greater.

The hydrophobicity of the chelator is such that it is capable of partitioning into the aggregates of the non-ionic detergent. In one embodiment, the chelator is capable of embedding into the aggregates of the non-ionic detergent.

In one embodiment, the hydrophobic chelator comprises at least 8 carbons (for example in a chain, or in a ring) and does not comprise charged groups.

In some embodiments, the hydrophobic chelator is 8-Hydroxyquinoline or a derivative thereof. Exemplary derivatives of 8-Hydroxyquinoline include, but are not limited to 2-methyl-8-hydroxyquinoline (CH3-HQ), 5,7-dichloro-2-methyl-8-hydroxyquinoline (Cl2-CH3-HQ), 5,7-dibromo-8-hydroxyquinoline (Br2-HQ), 5-sulfo-7-iodo-8-hydroxyquinoline (ferron) and 5-sulfo-8-hydroxyquinoline (SO3H-HQ).

In some embodiments, the hydrophobic chelator comprises a phenanthroline, for example a 1,10-Phenanthroline. Other phenanothrolines are also contemplated which have not been substituted with hydrophilic substituents.

Exemplary hydrophobic phenanthrolines include, but are not limited to bathophenanthroline, and N-(1,10-Phenanthrolin-5-yl)alkylamide), with the alkyl being from 1-carbon atoms in length. Exemplary N-(1,10-Phenanthrolin-5-yl)alkylamide) compounds include N-(1,10-Phenanthrolin-5-yl)methan amide) (Phen-C1), N-(1,10-Phenanthrolin-5-yl)ethanamide) (Phen-C2), N-(1,10-Phenanthrolin-5-yl)propanamide) (Phen-C3), N-(1,10-Phenanthrolin-5-yl)butan amide) (Phen-C4), N-(1,10-Phenanthrolin-5-yl)pentanamide) (Phen-C5), N-(1,10-Phenanthrolin-5-yl)hexanamide) (Phen-C6), N-(1,10-Phenanthrolin-5-yl)heptanamide) (Phen-C7), N-(1,10-Phenanthrolin-5-yl)octanamide) (Phen-C8), N-(1,10-Phenanthrolin-5-yl)nonanamide) (Phen-C9), N-(1,10-Phenanthrolin-5-yl)dec an amide) (Phen-C10).

In some such embodiments, the phenanthroline is selected from the group consisting of bathophenanthroline, N-(1,10-Phenanthrolin-5-yl)hexanamide) (Phen-6), N-(1,10-Phenanthrolin-5-yl)decanamide) (Phen-C10) and N-(1,10-Phenanthrolin-5-yl)octanamide) (Phen-C8).

Herein throughout, an “alkylamide” describes a —NH—C(═O)—R, wherein R is alkyl.

The term “alkyl” describes a saturated aliphatic hydrocarbon including straight chain and branched chain groups. Preferably, the alkyl group has 1 to 20 carbon atoms in length. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. The alkyl group may be substituted or unsubstituted. Substituted alkyl may have one or more substituents, whereby each substituent group can independently be, for example, alkyl, cycloalkyl, alkenyl, alkynyl, aryl, and heteroaryl. Additional substitutents may include, for example, hydroxyalkyl, trihaloalkyl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine, as long as the functionalities of the chelator are maintained.

In some embodiments, the phenanthroline is Phen-C10 or Phen-C8.

Additional examples of hydrophobic chelators include acidic organophosphorus chelators, for example DEHPA, EHEHPA and DTMPPA; neutral organophosphorus chelators, for example TBP and tri-n-octylphosphine oxide (TOPO), bifunctional organophosphorus chelators, for example CMPO and N,N,N′,N′-tetraoctyl-3-oxamentanediamide (TOGDA); basic chelators, for example tri-n-octylamine (TOA) and tricaprylmethylammonium chloride. Other chelators known to those of skill in the art may also be used, including hydroxyoximes, for example 5,8-diethyl-7-hydroxy-6-dodecane oxime and 2-hydroxy-5-nonylacetophenon oxime, crown ethers, for example di-t-butyl-dicyclohexano-18-crown-6, and dithiosemicarbazone.

According to some embodiments, the hydrophobic chelator is present in the medium (e.g. aqueous solution) at a concentration in the range of about 0.1% to about 10% (v/v), such as, for example, about 0.5% to about 10% (v/v), about 1% to about 10% (v/v) such as for example about 5%, about 6%, about 7%, about 8%, about 9%, or about 10% of 20 mM solution of chelator.

The iron salt of this aspect of the present invention comprises a monovalent anion including, but not limited to fluoride, bromide and chloride.

According to a specific embodiment, the iron salt is iron chloride.

In some embodiments, the metal ion is present in the medium (i.e. aqueous solution) at a concentration in the range of about 0.1% to about 10% (v/v), such as, for example, about 0.5% to about 10% (v/v), about 1% to about 10% (v/v), about 5%, about 6%, about 7%, about 8%, about 9%, or about 10% of 50 mM solution of metal ion (e.g. Fe²⁺).

The conditions of the incubation are such that aggregates are formed comprising the metal ion (e.g. Fe²⁺), the hydrophobic chelator and the non-ionic detergent.

Thus, for example, generation of aggregates is typically carried out at a temperature of about 0° C. to about 25° C. and more preferably from about 4° C. to about 25° C. The aggregates of this aspect of the present invention are typically between 10-500 nM, 10-200 nM, 1-100 mM or 10-100 mM.

The concentration of salt (e.g. NaCl) in the aggregates is typically, below 100 mM and more preferably below 50 mM. In one embodiment, the concentration of salt is below 40 mM, below 30 mM, below 20 mM, below 10 mM or even below 5 mM. Exemplary ranges include 20-100 mM, 20-50 mM, 0-50 mM, 0-40 mM, 0-30 mM, 0-25 mM, 0-20 mM. In one particular embodiment, the concentration of salt is about 25 mM.

In some embodiments, contacting the non-ionic detergent with a hydrophobic chelator is performed prior to contacting with a metal ion (e.g. Fe²⁺).

In other embodiments, contacting the non-ionic detergent with a hydrophobic chelator is performed concomitantly to contacting with a metal ion (e.g. Fe²⁺).

In still further embodiments, the hydrophobic chelator is contacted initially with the metal ion (e.g. Fe²⁺) and then subsequently with the non-ionic detergent.

Preferably, the aggregates that are formed are greater than 500 nM in diameter.

Preferably, the aggregates that are formed are greater than 1000 nM in diameter.

Preferably, the aggregates that are formed are greater than 2000 nM in diameter.

Typical contemplated ranges are between 500-3000 nM in diameter, 1000-3000 nM in diameter or 500-2000 nM in diameter.

Once aggregates are formed, they are contacted with the cell lysate under conditions that allow partitioning of the antibody (present in the cell lysate) into the aggregate.

Once this happens (seconds to hours—for example 5 minutes to 1 hour), precipitation of the complex may be facilitated by filtration.

Thus, according to another aspect of the present invention there is provided a method of isolating an antibody, the method comprising:
(a) contacting a hydrophobic chelator, a non-ionic detergent and metal ions so as to generate an aggregate comprising the hydrophobic chelator, the detergent and the metal ions;
(b) contacting the aggregate with a medium comprising the antibody under conditions that allow partitioning of the antibody into the aggregate; and subsequently
(c) filtering the medium comprising the aggregate, thereby isolating the antibody.

It will be appreciated that for this aspect of the present invention any metal ion is contemplated.

In some embodiments, the metal ion is a divalent metal ion.

In some embodiments, the divalent metal ion is selected from the group consisting of Zn²⁺, Fe²⁺, Mn²⁺, Ni²⁺ and Co²⁺. Preferably, the divalent metal ion Zn²⁺ or Fe²⁺.

The filters are selected according to the size of the aggregates.

In one embodiment, the filters are 0.2 micron filters, 0.22 micron filters or even 0.45 micron filters.

According to a particular embodiment, the filters are 0.1 micron filters.

Following the filtration, the antibody may be released from the pelleted complex i.e. solubilized.

Initially, the pellet may be washed—for example in a low salt solution (e.g. below 50 mM e.g. 20 mM NaCl solution).

Extraction may be effected with a buffer having a pH between 3-6, and more preferably between 3.8-5. In one embodiment, the buffer is a carboxylic buffer, examples of which include, but are not limited to sodium acetate and sodium citrate. An exemplary pH of sodium acetate is about pH 4.6.

In another embodiment, the buffer comprises an amino acid. In one embodiment, the buffer comprises a single amino acid. In another embodiment, the buffer comprises at least two amino acids.

In one embodiment, the amino acid is one which can competes for (i) hydrophobic interactions between the antibody side chains and the detergent aggregate (e.g. valine or isoleucine); (ii) ionic and/or H-bond interactions between the antibody side chains and the detergent aggregate (e.g. aspartic acid, glutamic acid or arginine); or (iii) metal chelation interactions between the antibody side chains and the detergent aggregate (e.g. histidine).

In a particular embodiment, the amino acid buffer is glycine, valine or isoleucine. In another embodiment, the amino acid buffer is isoleucine.

An exemplary pH of amino acid buffers is about pH 3.8 or pH 4.

The sample may be heated for a length of time that enhances extraction—for example (1-60 minutes), 1 minute, 5 minutes, 10 minutes. The temperature is selected such that it does not have an impact on the activity of the extracted antibody and does not cause the detergent aggregate to undergo dissolution. An exemplary temperature is between 25-35° C. According to a particular embodiment, the sample is heated for 5 minutes at 32° C.

To enhance the purity of the released antibody, salt may be added to the buffer (e.g. between 5-50 mM NaCl or 10-20 mM NaCl). To enhance the amount of antibody released from the complexed pellet, the present inventors contemplate using buffers which do not contain salt. It will be appreciated however, that the purity of the released antibody may then be compromised.

As mentioned, the present inventors contemplate reusing the metal chelator to purify additional antibodies as further described herein below.

Thus, according to another aspect of the present invention there is provided a method of preparing an aggregate, the method comprising:
(a) contacting a hydrophobic chelator, a non-ionic detergent and metal ions so as to generate a first aggregate comprising the hydrophobic chelator, the detergent and the metal ions;
(b) contacting the aggregate with a medium comprising an antibody under conditions that allow a first fraction of the antibody to partition into the aggregate;
(c) isolating the antibody from the aggregate;
(d) disassociating the aggregate;
(e) isolating the hydrophobic chelator; and subsequently
(f) contacting the hydrophobic chelator, a non-ionic detergent and metal ions so as to generate a second aggregate comprising the hydrophobic chelator, the detergent and the metal ions, thereby preparing the aggregate.

Steps (a), (b) and (c) have been described herein above, although it will be appreciated that step (b) can be facilitated by centrifugation (e.g. ultra-centrifugation), instead of (or together with) the filtration.

In order to dissociate the aggregate, the detergent is solubilized by adding salt to the medium (which comprises residual antibodies, that were not extracted during the purification process). Exemplary salts include NaCl (e.g. at concentrations between 0.25-1M) and Ammonium sulfate, AS. The complex may then be dissociated using a water-soluble chelator (e.g. EDTA or EGTA) that could compete with the hydrophobic chelator on binding to the metal ions. The chelator can optionally be added together with an alcohol (e.g. methanol). The solution is then heated to a temperature between 80 degrees and 100 degrees, for example 95 degrees for between 2-5 minutes.

Once disassociated, the hydrophobic chelator (e.g. bathophenanthroline) can be recrystallized so as to exclude any residual antibody which has not been removed during the first round of purification.

Recrystallization of bathophenanthroline is accomplished due to its extensive planar aromatic system. This inherent planarity and lipophilic nature of the chelator are ideal for promoting pi-pi (Π-Π) stacking between bathophenanthrolines and thus represent the driving force for its rapid crystal growth in aqueous media. The presence of EDTA does not interfere with the above, since EDTA is charged and as such, is repelled from the highly lipophilic faces of the growing crystals.

The recrystallized, purified hydrophobic chelator can then be reused to generate additional aggregates, which in turn can be used to aid in the purification of additional antibodies.

Depending on the intended use of the antibody that is isolated and optionally solubilized, the protein (either membrane or cytosolic) or agent that is bound thereto, may be subjected to further purification steps. This may be effected by using a number of biochemical methods which are well known in the art. Examples include, but are not limited to, fractionation on a hydrophobic interaction chromatography (e.g. on phenyl sepharose), ethanol precipitation, isoelectric focusing, reverse phase HPLC, chromatography on silica, chromatography on heparin sepharose, anion exchange chromatography, cation exchange chromatography, chromatofocusing, SDS-PAGE, ammonium sulfate precipitation, hydroxylapatite chromatography, gel electrophoresis, dialysis, viral inactivation (e.g. viral filtration) and ultrafiltration.

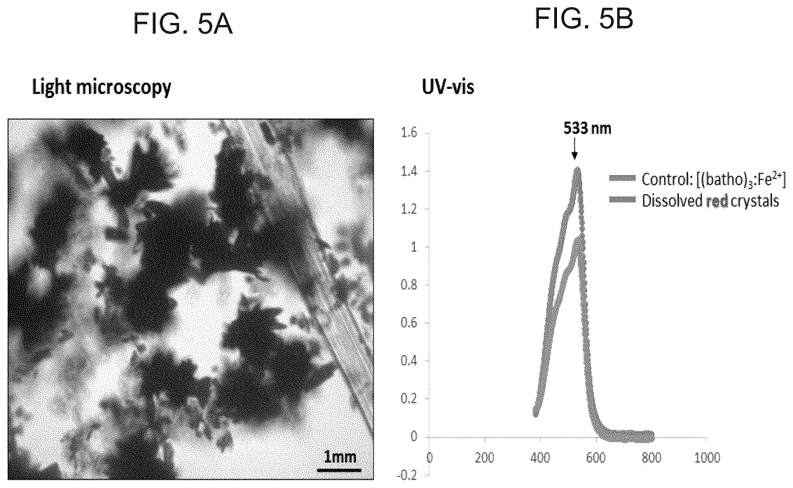

Examples of additional purification steps (and the order they may be carried out) are summarized in FIG. 5B.

Anion-exchange chromatography is a process that separates substances based on their charges using an ion-exchange resin containing positively charged groups, such as diethyl-aminoethyl groups (DEAE). In solution, the resin is coated with positively charged counter-ions (cations). Anion exchange resins will bind to negatively charged molecules, displacing the counter-ion.

Cation-exchange chromatography is a process that separates substances based on their charges using an ion-exchange resin containing negatively charged groups, such as carboxymethyl (CM), sulfoethyl(SE), sulfopropyl(SP), phosphate(P) and sulfonate(S). In solution, the resin is coated with negatively charged counter-ions (anions). Cation exchange resins will bind to positively charged molecules, displacing the counter-ion.

The phrase “viral inactivation”, as used herein, refers to a decrease in the activity of adventitious enveloped viruses in a particular sample (“inactivation”). Such decreases in the activity of enveloped viruses can be on the order of about 3 log reduction factor (LRF) preferably of about 4 LRF, more preferably of about 5 LRF, even more preferably of about 6 LRF.

Any one or more of a variety of methods of viral inactivation can be used including heat inactivation (pasteurization), pH inactivation, solvent/detergent treatment, UV and γ-ray irradiation and the addition of certain chemical inactivating agents such as (3-propiolactone or e.g., copper phenanthroline as in U.S. Pat. No. 4,534,972, the entire teaching of which is incorporated herein by reference.

Methods of pH viral inactivation include, but are not limited to, incubating the mixture for a period of time at low pH, and subsequently neutralizing the pH. In certain embodiments the mixture will be incubated at a pH of between about 2 and 5, preferably at a pH of between about 3 and 4, and more preferably at a pH of about 3.6.

The pH of the sample mixture may be lowered by any suitable acid including, but not limited to, citric acid, acetic acid, caprylic acid, or other suitable acids. The choice of pH level largely depends on the stability profile of the antibody product and buffer components. It is known that the quality of the target antibody during low pH virus inactivation is affected by pH and the duration of the low pH incubation. In certain embodiments the duration of the low pH incubation will be from 0.5 hr to 2 hr, preferably 0.5 hr to 1.5 hr, and more preferably the duration will be about 1 hr. Virus inactivation is dependent on these same parameters in addition to protein concentration, which may limit inactivation at high concentrations.

Thus, the proper parameters of protein concentration, pH, and duration of inactivation can be selected to achieve the desired level of viral inactivation.

In certain embodiments viral filtration is performed. This can be achieved via the use of suitable filters. A non-limiting example of a suitable filter is the Ultipor DV50™ filter from Pall Corporation. In certain embodiments, alternative filters are employed for viral inactivation, such as, but not limited to, Sartorius filters, Viresolve™ filters (Millipore, Billerica, Mass.); Zeta Plus VR™ filters (CUNO; Meriden, Conn.); and Planova™ filters (Asahi Kasei Pharma, Planova Division, Buffalo Grove, 111.).

Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). A preferred filtration process is Tangential Flow Filtration as described in the Millipore catalogue entitled “Pharmaceutical Process Filtration Catalogue” pp. 177-202 (Bedford, Mass., 1995/96). Ultrafiltration is generally considered to mean filtration using filters with a pore size that allow transfer of protein with average size of 50 kDa (for example) or smaller. By employing filters having such small pore size, the volume of the sample can be reduced through permeation of the sample buffer through the filter while antibodies are retained behind the filter.

Diafiltration is a method of using ultrafilters to remove and exchange salts, sugars, and non-aqueous solvents, to separate free from bound species, to remove low molecular-weight material, and/or to cause the rapid change of ionic and/or pH environments. Microsolutes are removed most efficiently by adding solvent to the solution being ultrafiltered at a rate approximately equal to the ultratfiltration rate. This washes microspecies from the solution at a constant volume, effectively purifying the retained antibody. In certain embodiments of the present invention, a diafiltration step is employed to exchange the various buffers used in connection with the instant invention, optionally prior to further chromatography or other purification steps, as well as to remove impurities from the antibody.

In one embodiment, the antibody which is isolated is crystallized.

As used herein the term “crystallizing” refers to the solidification of the molecule of interest so as to form a regularly repeating internal arrangement of its atoms and often external plane faces.

Several crystallization approaches which are known in the art can be applied to the sample in order to facilitate crystallization of the molecule of interest. Examples of crystallization approaches include, but are not limited to, the free interface diffusion method [Salemme, F. R. (1972) Arch. Biochem. Biophys. 151:533-539], vapor diffusion in the hanging or sitting drop method (McPherson, A. (1982) Preparation and Analysis of Protein Crystals, John Wiley and Son, New York, pp 82-127), and liquid dialysis (Bailey, K. (1940) Nature 145:934-935).

Presently, the hanging drop method is the most commonly used method for growing macromolecular crystals from solution; this approach is especially suitable for generating protein crystals. Typically, a droplet containing a protein solution is spotted on a cover slip and suspended in a sealed chamber that contains a reservoir with a higher concentration of precipitating agent. Over time, the solution in the droplet equilibrates with the reservoir by diffusing water vapor from the droplet, thereby slowly increasing the concentration of the protein and precipitating agent within the droplet, which in turn results in precipitation or crystallization of the protein.

According to still another aspect there is provided a method of isolating an antibody fragment of interest comprising:
(a) contacting a hydrophobic chelator, a non-ionic detergent and metal ions so as to generate an aggregate comprising said hydrophobic chelator, said non-ionic detergent and said metal ions;
(b) contacting said aggregate with a medium comprising:
(i) an antibody fragment which comprises an Fc region; and
(ii) an antibody fragment which is devoid of an Fc region,
wherein said contacting is effected under conditions that allow selective partitioning of one of said fragments (i) or (ii) into said aggregate so as to generate an antibody fragment-enriched aggregate and an antibody fragment-enriched medium; and
(c) isolating said antibody fragment of interest from said fragment-enriched aggregate or from said fragment-enriched medium, thereby isolating the antibody fragment of interest.

Hydrophobic chelators, non-ionic detergents and metal ions are described herein above and methods of generating aggregates therefrom are also described herein above.

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly.

These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used.

The method according to this aspect of the present invention comprises isolating a first antibody fragment which comprises the Fc region from a second antibody fragment which is devoid of the Fc region.

The term “Fc region” refers to a C-terminal region of an antibody heavy chain that contains at least a portion of the constant region.

The term includes native sequence Fc regions and variant Fc regions.

In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat, E. A. et al., Sequences of Proteins of Immunological Interest, 5th ed., Public Health Service, National Institutes of Health, Bethesda, Md. (1991), NIH Publication 91-3242.

The Fc comprising fragment is typically more hydrophobic and/or has an enhanced ability to bind to metal ions that the antibody fragment which is devoid of the Fc region.

For example, the Fc region may have at least one of the following properties:
(i) contains at least 20, 21, 22, 23, 24, 25, 26, 27, 28, 29 or 30 glutamate residues and/or at least 8, 9, 10, 11, 12 or 13 histidine residues (enhances metal binding);
(ii) contains at least 40, 41, 42, 43, 44 or 45 valines and/or at least 30, 31, 32, 33, 34, 35, 36, 37, 38, 39 or 40 proline residues (enhances hydrophobicity);
(iii) Contains less than 35, 34, 33 or 32 threonines and/or less than 50, 49, 48, 47, 46, 45, 44, 43, 42, 41 or 40 serines (reduces hydrophilicity).

Suitable antibody fragments which do not comprise an Fc region contemplated by the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, an Fd fragment, and antibody fragments comprising essentially whole variable regions of both light and heavy chains such as an Fv, a single chain Fv (scFv), a disulfide-stabilized Fv (dsFv), an Fab, an Fab′, and an F(ab′)2.

Exemplary fragments which are devoid of the Fc region are further defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule.
(iii) disulfide-stabilized Fv (“dsFv”), a genetically engineered antibody including the variable region of the light chain and the variable region of the heavy chain, linked by a genetically engineered disulfide bond.
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and
(vii) Single domain antibodies or nanobodies are composed of a single VH or VL domains which exhibit sufficient affinity to the antigen.

According to a particular embodiment, the antibody fragment which is devoid of the Fc region is the Fab fragment and/or the F(ab)₂ fragment.

In one embodiment, the Fc fragment is selectively retained in the aggregate over the fragment which is devoid of the Fc region.

In this scenario the antibody fragment-enriched medium is enriched in the fragment which is devoid of the Fc region over the fragment which comprises the Fc region.

In one embodiment, the antibody fragments in the medium are derived from the same full length antibody.

Conditions for selective partition of the Fc fragment into the aggregate include a pH between 5-9. According to a particular embodiment, the pH is about 7-8.

Incubation time may be between 1-60 minutes. According to a particular embodiment, the incubation time is between 5-10 minutes.

Salt concentration of the medium may be between 1-200 mM, e.g. about 15 mM NaCl.

Temperature of the incubation may be between 4-37° C., e.g. between 15-37° C.

Conditions for the selective release of the fragment which is devoid of the Fc region include a temperature below 32° C., e.g. between 25° C.-32° C.

Conditions for the selective release of the Fc fragment include a pH below 3 (e.g. between 2.5 and 3). Conditions for the selective release of the fragment devoid of the Fc region include a pH above 3.8 (for example between 3.8 and 4.5).

Conditions for the selective release of the fragment devoid of the Fc region include a salt concentration above 75 mM.

The agents used for purifying the antibody may be provided as a kit.

It is expected that during the life of a patent maturing from this application many relevant hydrophobic chelators will be developed and the scope of the term hydrophobic chelator is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Purification of Antibody by Filtration

Materials

Bovine serum albumin, 4,4′-dinonyl-2,2′dipyridyl, Protein A HP Spin-Trap, Protein AB Spin-Trap, isoleucine, FeSO₄:heptahydrate, NaCl, NiBr₂, ZnCl₂, MnCl₂, CuSO₄, MgCl₂, CaCl₂, Tween-20 (Polysorbate 20), Ex-CELL 610-HSF medium, ethylenediaminetetraacetic acid (EDTA), 1,2-dihydroxybenzene (catechol), histidine (His), and imidazole were all purchased from Sigma-Aldrich; glycine (Bio-lab), PM2700 ExelBand 3-color Broad range protein marker (Smobio, Taiwan), bathophenanthroline (GFS chemicals) (batho), 4′-diphenyl-2,2′dipyridyl (Santa Cruz Biotechnology, CA, USA), human IgG (hIgG) (Lee-Bio-solutions, 340-21, MO, USA), mouse IgG (Equitech, SLM66, TX, USA).

Methods

### Preparation of [Tween-20:Chelator:M²⁺] Aggregates

Tween-20 aggregates were obtained by mixing equal volumes of medium #1 and medium #2 as follows. For medium #1, 30 μL of 20 mM amphiphilic chelator in methanol was added to 270 μL of 0.25 mM Tween-20 in DDW with vigorous vortexing to achieve a final volume of 300 μL. An equal volume of medium #2, containing FeSO₄, CuSO₄, NiBr₂, ZnCl₂, MnCl₂, MgCl₂ or CaCl₂ (all 1 mM) in 20 mM NaCl, was then added with vigorous vortexing. After 5 min incubation at room temperature, the mixture was centrifuged (13K, 2 min.), the supernatant discarded and the resulting pellet washed with 20 mM NaCl in DDW and analyzed. In order to control pH, Tween-20 aggregates were prepared as described above but with the addition of pH buffers: 50 mM acetic acid (pH 3, 4, and 5) or 50 mM Tris(hydroxymethyl)aminomethane (Tris) (pH 6, 7, 8, or 9). Chelators phen-C10 and 4,4′diphenyl-2,2′-dippyridyl (dipyridyl phenyl) were tested with Ni²⁺, Fe²⁺, Zn²⁺ and Mn²⁺ (all under identical conditions).

Antibody Capture Using Conjugated Tween-20 Micelles

Freshly prepared, conjugated Tween-20 micellar aggregates were resuspended in 100 μL serum-free medium (Ex-CELL 610-HSF, NaHCO₃ buffer: pH-6.72) containing 5% PEG-6000, the target IgG (1 mg/mL) and BSA (0.5 mg/mL). After 5-10 min incubation at room temperature, centrifugation (13K, 2 min.) was applied, the supernatant discarded and pellets were briefly washed with 100 μL of cold 20 mM NaCl. An additional centrifugation step followed (13K, 2 min.), the supernatant was removed and pellets were analyzed by SDS-PAGE.

General Extraction Protocol of IgG's from Conjugated Tween-20 Micellar Aggregates

Pellets containing target IgG were incubated with 100 μL of 50 mM glycine (pH 3.8), 30 mM NaCl for 5 minutes at 32° C. Centrifugation followed (13K, 2 minutes) and the supernatant was carefully removed for analysis on SDS-PAGE gels.

### Preparation of [Tween-20:Batho:Fe²⁺] Aggregates at Different pH

Tween-20 aggregates were prepared as described above but in the presence of pH buffers: 50 mM acetic acid (for pH 3, 4, and 5) or 50 mM Tris(hydroxymethyl)aminomethane (Tris) (for pH 6, 7, 8, or 9).

Dynamic Light Scattering (DLS)

The intensity-weighted size distributions of extracted human and mouse IgG samples were determined using the autocorrelation protocol of the Nanophox instrument (Sympatec GmbH, Germany).

Densitometry

Bands on Coomassie stained gels were quantified using the ImageJ (NIH) program.

Results

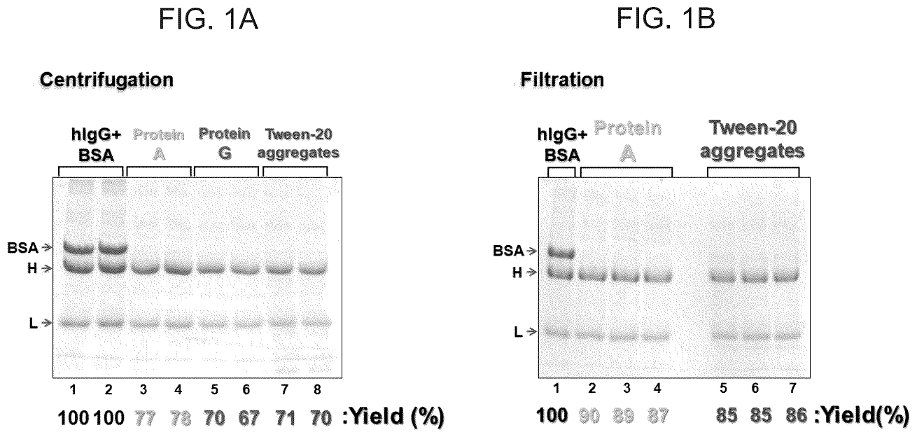

Current findings show that, purification of IgG's with: [Tween-20:bathophenanthroline:Fe²⁺] detergent aggregates can rely on filtration rather than on centrifugation (FIGS. 1A-B). Under these circumstances, IgG's are relatively pure and lead to overall greater recovery yields (FIGS. 1A-B).

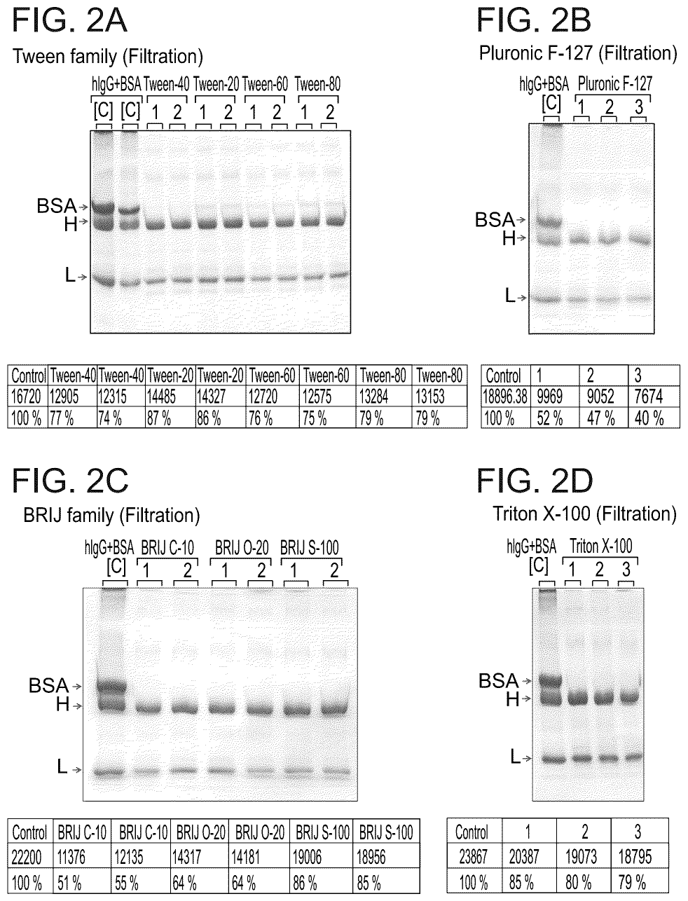

Implementation of the purification protocol to other members of the Tween family and to other surfactant families (e.g. Brij, Triton, Pluronic) show that IgG purification is accomplished (FIGS. 2A-D).

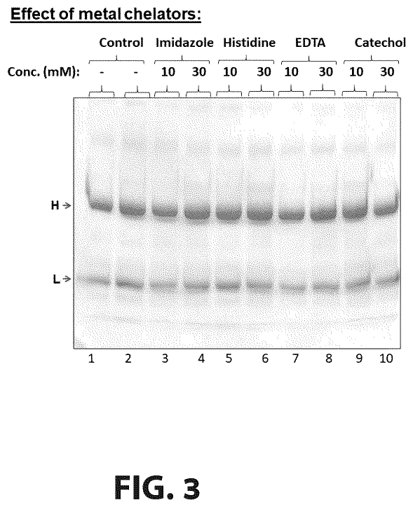

It has been found that purification can be performed in the presence of different chelators (e.g. EDTA, Catechol, Histidine, imidazole) and lead to relatively pure antibody and good recovery yields (FIG. 3).

### Example 2

### Chelator Recycling

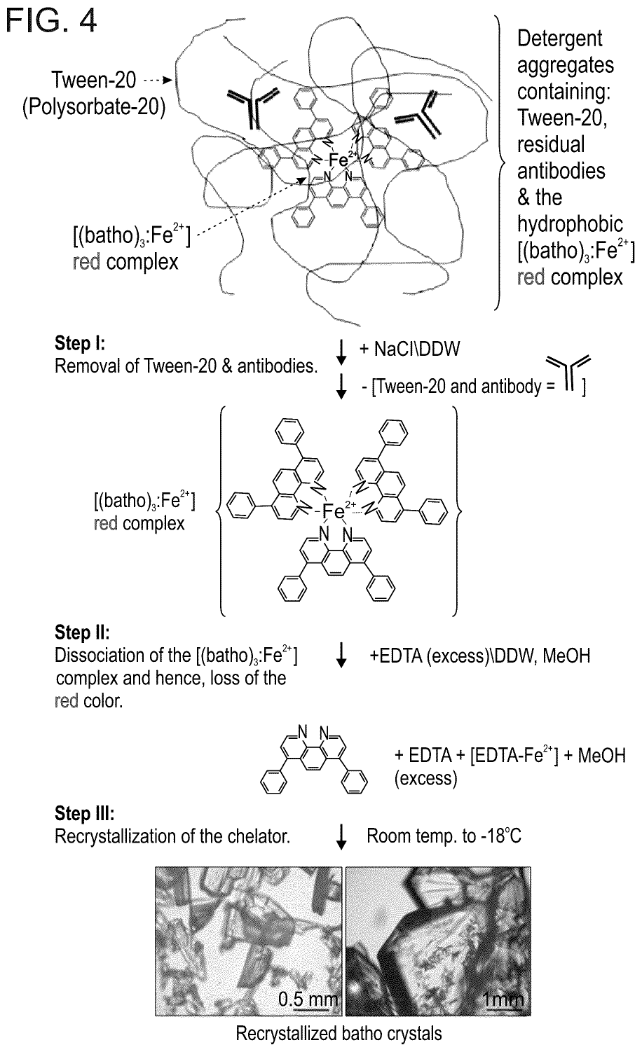

The general strategy for recycling the chelator: bathophenanthroline (batho) via recrystallization is illustrated in FIG. 4.

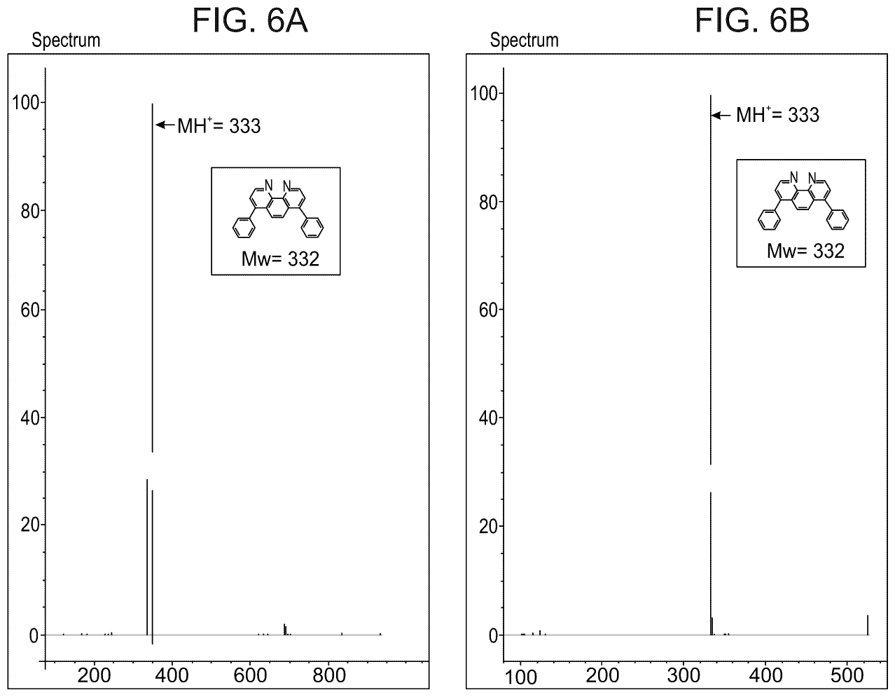

In brief, pellets comprised of Tween-20 (polysorbate-20), the red hydrophobic complex: [(batho)₃:Fe²⁺] and residual antibodies (that were not extracted during the purification process), were resuspended in 0.25-1M NaCl to solubilize the detergent (Tween-20) and IgG's while keeping the majority of the highly hydrophobic [(batho)₃:Fe²⁺] red complex, insoluble. (FIG. 4—Step I) Following a short spin, the supernatant was discarded and the resulting pellets were found to be comprised of red crystals (FIG. 5A) that exhibited identical absorption (λₘₐₓ=533 nm) to the freshly prepared [batho)₃:Fe²⁺] red complex. (FIG. 5B). Addition of MeOH and molar excess of EDTA (pH 7) allowed total dissolution of the red crystals and loss of the solution red color (FIG. 4—Step II), presumably due to the presence of EDTA. The latter, is a strong water-soluble chelator that could compete with batho on binding to Fe²⁺ ions thereby leading to the dissociation of the: [batho)₃:Fe²⁺] complex and generation of the colorless [EDTA-Fe²⁺] complex. Slow cooling of the solution promoted the growth of colorless crystals (FIG. 3—Step III) comprised of the regenerated chelator. (FIGS. 6A-B).

Recycling Protocol:
1. Add 400 μL of 0.5 M NaCl to the resulting pellet after the IgG extraction step.
2. Vortex for 1 minute and apply centrifugation (13K, 5 minutes, room temp).
3. Remove the supernatant.
4. Add MeOH (50 μL) and 0.2 M EDTA (200 μL, pH 7) in DDW.
5. Incubate for 2-5 minutes at 95° C. until the solution becomes colorless and clear.
6. Add 800 μL of DDW.
7. Allow the system to cool to room temperature (e.g. 15 minutes) and then, to −4° C. or −18° C. (if needed).
8. Apply centrifugation and exclude the supernatant (13K, 5 min., room temp.)
9. Wash crystals with cold DDW (500 μL), centrifuge and discard the water.

Quantification of Recycling Efficiency:
1. Dissolve crystals with MeOH to a final known volume.
2. Add excess Fe²⁺ (e.g. 100 mM).
3. Measure absorbance at 530-533 nm (representing the: λₘₐₓ of the [(batho)₃:Fe²⁺] red complex).

### Example 3

### Effect of Counter Ion or Temperature on IgG Purification Using Tween-20 Aggregates

Materials and Methods

Preparation of detergent aggregates: Detergent aggregates were obtained by mixing equal volumes of medium A and B as follows: medium A was prepared by the addition of 20 μL of the hydrophobic chelator bathophenanthroline (20 mM in methanol) to 180 μL of: 0.25 mM; 0.215 mM; 0.062 mM; 0.062 mM; 0.1 mM; 0.09 mM; 0.05 mM; 0.1 mM and 0.5 mg/mL of Tween-20, Tween-40, Tween-60, Tween-80, Brij C-10, Brij 0-20, Brij S-100, Triton X-100 and Pluronic F-127 in DDW, respectively, with vigorous vortexing to a final volume of 200 μL. An equal volume of medium B, containing 1 mM FeSO₄ in 20 mM NaCl was then added to medium A with vigorous vortexing.

Purification of hIgG and mouse IgG with detergent aggregates: Freshly prepared aggregates were resuspended in 100 μL of serum-free medium (Ex-CELL 610-HSF) containing: 4% PEG-6000, the target IgG (1 g/mL) and BSA (0.25 mg/mL). After 5 minutes of incubation at room temperature, centrifugation (14K, 2 min.) was applied, the supernatant discarded and pellets were briefly washed with 100 μL of cold 20 mM NaCl. An additional centrifugation step followed (14K rpm, 2 min.), the supernatant was removed, and pellets were analyzed by SDS-PAGE.

Extraction of IgG's from detergent aggregates: Detergent aggregates containing the target IgG were incubated with 100 μL of: 50 mM Gly, 30 mM NaCl at pH 3.8 for 5 minutes at room temperature. Centrifugation followed (13K rpm, 2 min.) and the supernatant was carefully removed for further analysis.

Results

A greater IgG recovery yields was obtained when Tween-20 aggregates were prepared with ferrous chloride (FeCl₂) rather than with ferous sulfate (FeSO₄) (FIG. 7A). Moreover, the efficiency of the antibody “capturing step” improves at low temperature (FIG. 7B).

### Example 4

### Purification of Antibody Fragments with Engineered Micelles

Materials and Methods

Papain digestion: Antibody digestion was performed using the FragIT (Genovis) kit in a total volume of 100 μL containing: 20 mM NaCl, 10 NaPi (pH 7.4) and 0.5 mg (total amount) of the target mAb (CB72.3 Sarto QFT). After 3 hours of incubation at 37° C., centrifugation (200×g, 1 min) was applied and the sample was either used immediately or stored at −20° C.

Preparation of Brij S-100 detergent aggregates: Detergent aggregates were obtained by mixing equal volumes of medium A and B as follows: Medium A was prepared by the addition of 30 μl of the hydrophobic chelator bathophenanthroline (20 mM in methanol) to 270 μl of 0.05 mM Brij S-100 in DDW with vigorous vortexing. An equal volume of Medium B, containing 1 mM FeCl₂ in 20 mM NaCl was then added to medium A with vigorous vortexing. After 5 minutes of incubation at room temperature 23 μl of 5 M of NaCl were added and the system was further incubated for additional 5 minutes at 4° C. A short spin followed (13K, 5 minutes), the supernatant discarded and the aggregates were washed with 100 μl of DDW. Repetition of the spin resulted in red Brij S-100 aggregates that were used for purification of antibody fragments.

Purification protocol: Papain digested mAb (67 μl) were mixed with hybridoma serum free media (33 μl) and incubated for 10 minutes at room temperature with freshly prepared Brij S-100 aggregates (100 μl), Centrifugation was applied (13K, 5 minutes) and the supernatant was analyzed by SDS-PAGE.

Results

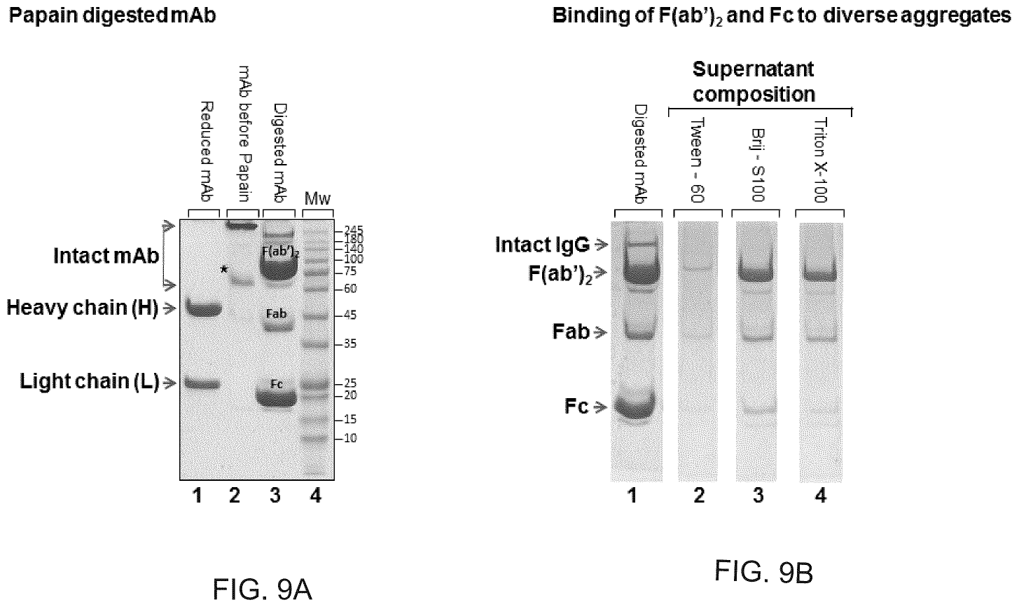

Digestion of the mAb by papain led to the two expected major fragments: F(ab′)₂ (˜75-100 KDa) and Fc (˜20 KDa) (FIG. 9A—lane 3). Additional minor bands, one at ˜40-45 KDa was assumed to represent the reduced Fab domain (FIG. 2A—lane 3).

An initial screen of the above and exemplary detergents representative of three different detergent types, illustrate preferential binding of the Fc domain (see for example Tween-60, Brij-C10, Brij-S100 and Triton X-100 captured preferentially the Fc domain (FIG. 9B).

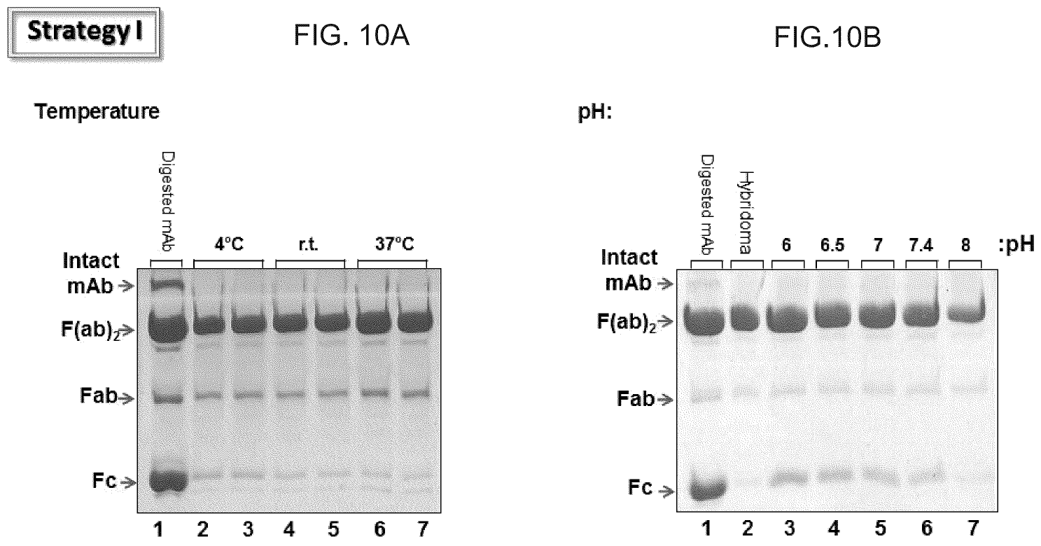

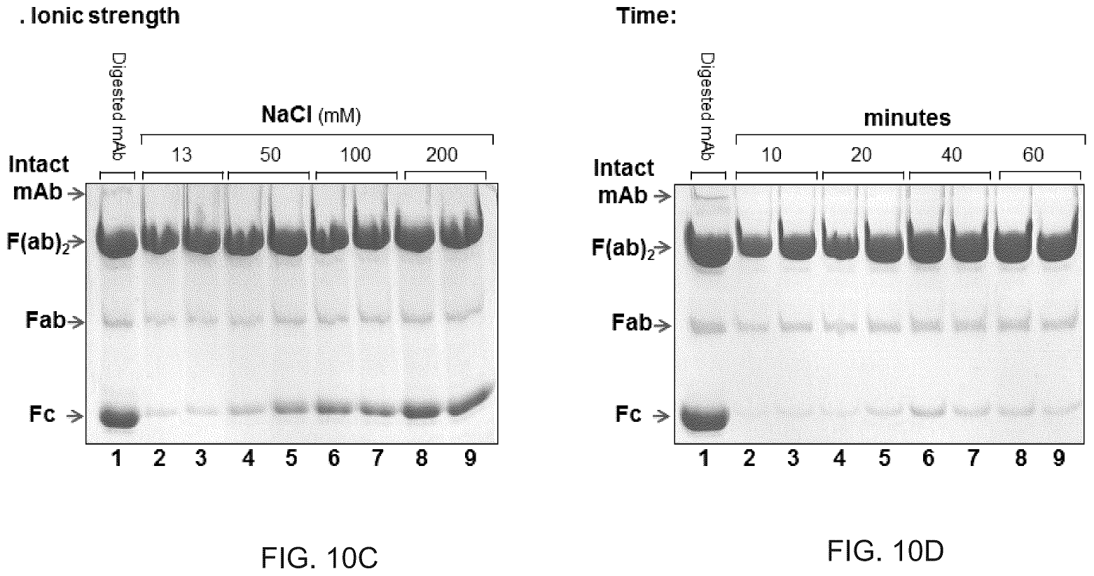

Brij-S100 was selected for further testing in a variety of working conditions, first of which was temperature during the binding step (FIG. 10A). It was found that incubation of Brij-S-100 aggregates with the fragment mixture at 37° C. (rather than 4° C. or room temperature) leads to the highest amount of Fab and F(ab′)₂ in the supernatant (FIG. 10A, lanes 6-7) without parallel increase in the amount of the Fc domain (FIG. 10A, lanes 2-5).

At higher pH (pH 8) no Fc was observed in the supernatant but process efficiency decreased dramatically (FIG. 10B, lane 7).

In addition to the pH, ionic strength was found to represent an additional important parameter for regulation of the Fc domain in the supernatant (FIG. 10C). At low ionic strength (e.g. 13 mM NaCl) and pH 7.4 the amount of Fc was minimal. The increase in the presence of Fc was found to be correlated with the increase in salt (FIG. 10C, lanes 8-9).

Optimal incubation time was ˜40 minutes (FIG. 10D, lanes 6-7) leading to very high recovery yields,

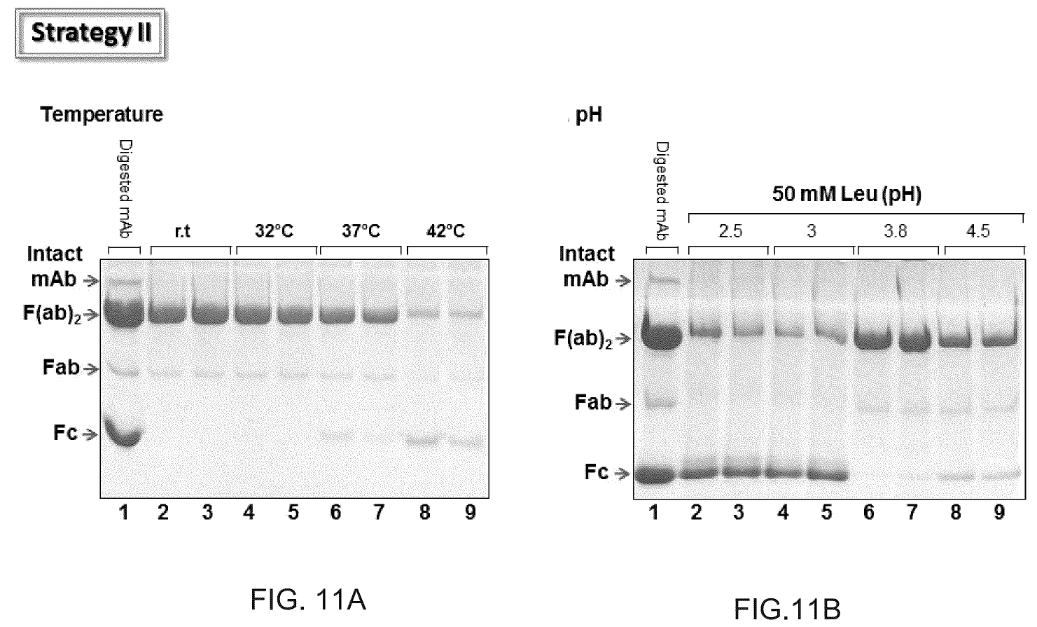

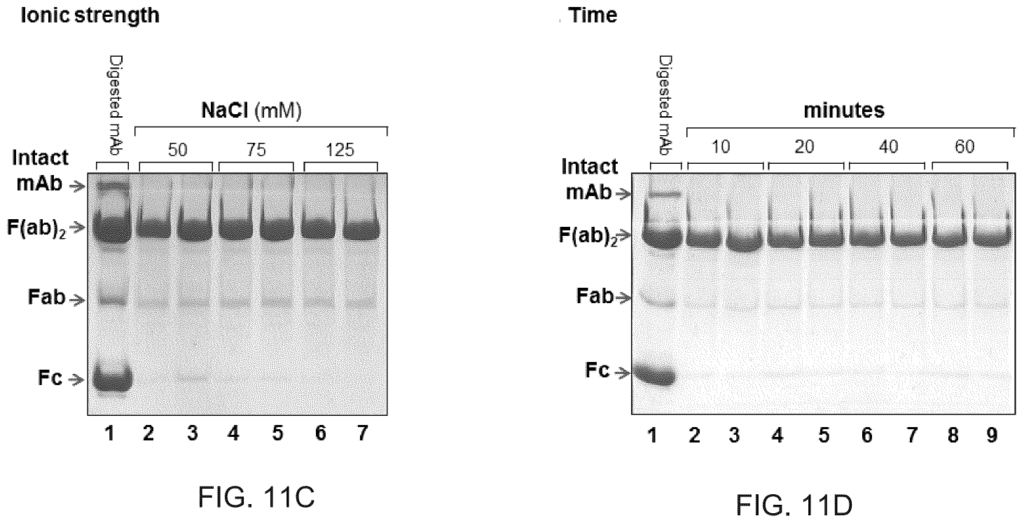

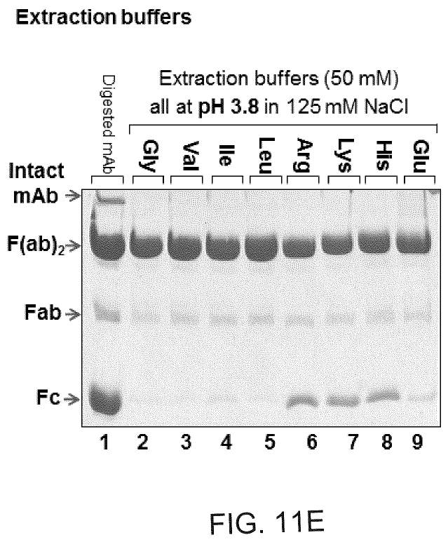

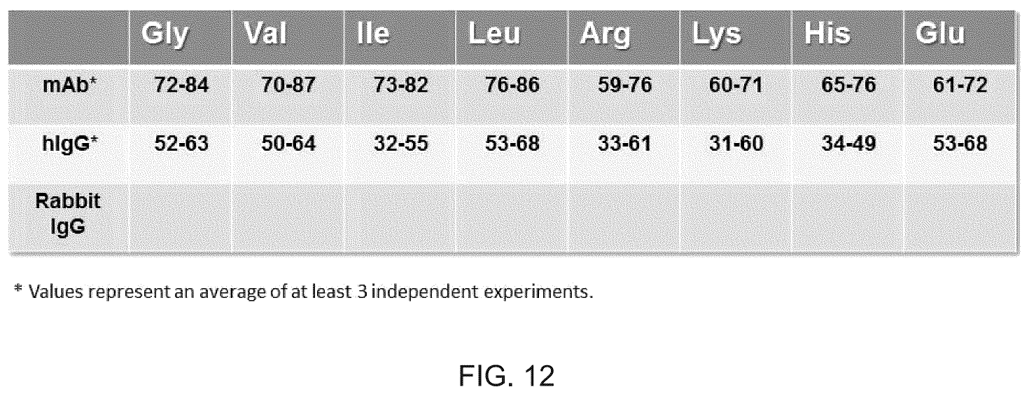

FIGS. 11A-D illustrate different conditions that can be used for selective release of antibody fragments.

In order to understand the greater binding affinity of the Fc fragment to diverse detergent aggregates, relative to the F(ab′)₂ domain, the amino acid sequence of 10 IgGs was analyzed.

The results indicate that, while the Fc domain is richer in residues capable of participating in metal chelation (Glu+66%, His+85%) and 2 hydrophobic amino acids (Val+21%, Pro+70%), the Fab domain contains a greater number of aliphatic hydroxylic side chains, in particular: Thr (+22%) and Ser (+60%).

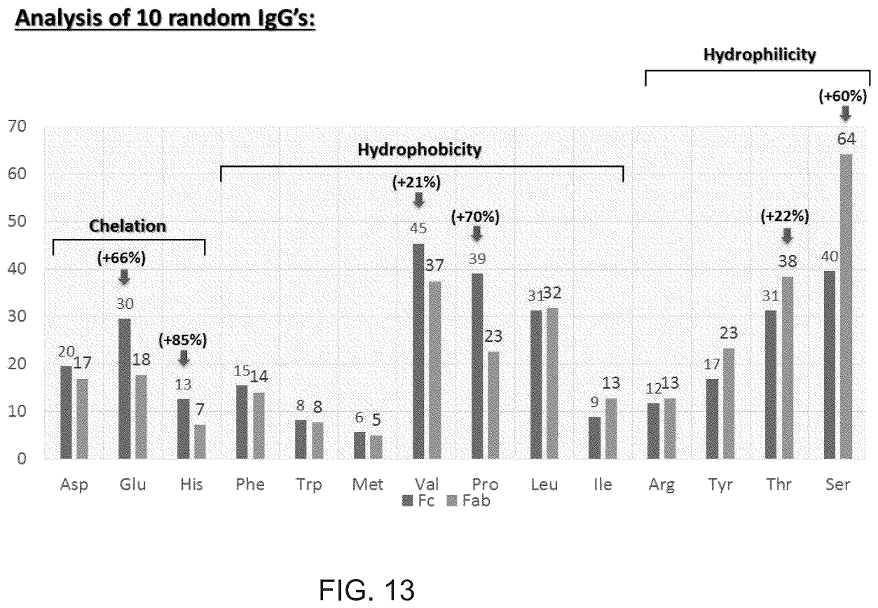

These findings suggest that, the Fc domain has a greater probability in (a) forming interaction with the [(bathophenathroline)₃:Fe²⁺] amphiphilic complex via the excess of imidazole (His) and carboxylate (Glu) side groups and (b) greater number of VDW interactions via the Val+Pro residues. At the same time, the Fab arms may be more hydrophilic (due to the excess in Ser+Thr amino acids) explaining their lower tendency towards adhering to the hydrophobic detergent aggerates (summarized in FIG. 13).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of isolating an antibody, the method comprising:
(a) contacting a hydrophobic chelator, a non-ionic detergent and an iron salt, so as to generate an aggregate comprising said hydrophobic chelator, said detergent and iron ions of said iron salt, said iron salt being selected from the group consisting of iron chloride, iron bromide and iron fluoride; and
(b) contacting said aggregate with a medium comprising the antibody under conditions that allow partitioning of the antibody into said aggregate, thereby isolating the antibody.

2. The method of claim 1 further comprising filtering said medium comprising said aggregate.

3. The method of claim 1, wherein said iron salt is iron chloride.

4. A method of isolating an antibody, the method comprising:
(a) contacting a hydrophobic chelator, a non-ionic detergent and metal ions so as to generate an aggregate comprising said hydrophobic chelator, said detergent and said metal ions;
(b) contacting said aggregate with a medium comprising the antibody under conditions that allow partitioning of the antibody into said aggregate; and subsequently
(c) filtering said medium comprising said aggregate, thereby isolating the antibody.

5. A method of preparing an aggregate, the method comprising:
(a) contacting a hydrophobic chelator, a non-ionic detergent and metal ions so as to generate a first aggregate comprising said hydrophobic chelator, said detergent and said metal ions;
(b) contacting said aggregate with a medium comprising an antibody under conditions that allow a first fraction of said antibody to partition into said aggregate;
(c) isolating said antibody from said aggregate;
(d) disassociating said aggregate;
(e) isolating said hydrophobic chelator; and subsequently
(f) contacting said hydrophobic chelator, a non-ionic detergent and metal ions so as to generate a second aggregate comprising said hydrophobic chelator, said detergent and said metal ions, thereby preparing the aggregate.

6. The method of claim 5, wherein said disassociating said aggregate comprises contacting said aggregate with a water soluble chelator under conditions that allows disassociation of said aggregate.

7. The method of claim 6, wherein said water soluble chelator comprises EDTA or EGTA.

8. The method of claim 5, wherein said isolating said antibody from said aggregate comprises filtering said medium comprising said aggregate.

9. The method of claim 1, wherein said aggregate has a diameter greater than 500 nM.

10. (canceled)

11. The method of claim 1, wherein said medium comprises a cell lysate.

12-13. (canceled)

14. The method of claim 1, wherein said medium comprises serum albumin.

15. (canceled)

16. The method of claim 1, wherein said conditions of step (b) comprise having a level of salt below 100 mM.

17. The method of claim 1, further comprising solubilizing said antibody following step (b).

18. The method of claim 5, wherein said isolating said antibody comprises solubilizing said antibody.

19. The method of claim 17, wherein said solubilizing is effected with a buffer having a pH between 3-6 wherein said buffer:
(i) further comprises a salt;
(ii) is a carboxylic buffer; or
(iii) comprises an amino acid.

20. The method of claim 17, wherein said solubilizing is effected with a buffer having a pH between 3.8 and 4.

21-23. (canceled)

24. The method of claim 19, wherein said carboxylic buffer is selected from the group consisting of isoleucine, valine, glycine and sodium acetate.

25. A method of isolating an antibody fragment of interest comprising:
(a) contacting a hydrophobic chelator, a non-ionic detergent and metal ions so as to generate an aggregate comprising said hydrophobic chelator, said detergent and said metal ions;
(b) contacting said aggregate with a medium comprising:
(i) an antibody fragment which comprises an Fc region; and
(ii) an antibody fragment which is devoid of an Fc region,
wherein said contacting is effected under conditions that allow selective partitioning of one of said fragments (i) or (ii) into said aggregate so as to generate an antibody fragment-enriched aggregate and an antibody fragment-enriched medium; and
(c) isolating said antibody fragment of interest from said fragment-enriched aggregate or from said fragment-enriched medium, thereby isolating the antibody fragment of interest.

26. The method of claim 25, wherein said conditions comprise a pH of between 5-9.

27. The method of claim 25, wherein said pH is between 7-8.

28. The method of claim 1, wherein said non-ionic detergent is a polysorbate surfactant.

29. The method of claim 28, wherein said polysorbate surfactant is selected from the group consisting of polysorbate 20, polysorbate 40, polysorbate 60 and polysorbate 80.

30. The method of claim 25, wherein said non-ionic detergent belongs to a family selected from the group consisting of the Tween family, the Brj family and the Triton family.

31. The method of claim 25, wherein said non-ionic detergent is selected from the group consisting of Tween-60, Brj-S100 and Triton-X-100.

32. (canceled)

33. The method of claim 1, wherein said hydrophobic chelator comprises a phenanthroline.

34. (canceled)

35. The method of claim 33, wherein said phenanthroline is selected from the group consisting of bathophenanthroline, N-(1,10-Phenanthrolin-5-yl)hexanamide) (Phen-6), N-(1,10-Phenanthrolin-5-yl)decanamide) (Phen-C10) and N-(1,10-Phenanthrolin-5-yl)octanamide) (Phen-C8).

36. (canceled)

37. The method of claim 4, wherein said metal ions are divalent metal ions.

38. The method of claim 37, wherein said divalent metal ions are selected from the group consisting of Zn²⁺, Fe²⁺, Mn²⁺, Ni²⁺ and Co²⁺.

39-42. (canceled)

43. The method of claim 1, wherein said hydrophobic chelator is present in said medium at a concentration in the range of about 0.1% to about 10% (v/v).

44. (canceled)

45. The method of claim 1, wherein said iron ions are present in said medium at a concentration in the range of about 0.1% about 10% (v/v).

46-54. (canceled)
